(19) United States
(12) Patent Application Publication
(10) Pub. No.: US 2026/0024546 A1
(43) Pub. Date: Jan. 22, 2026

(54) DEPRESSION DETECTION SYSTEM, HOST DEVICE, COMPUTER-READABLE STORAGE MEDIUM, AND EVALUATION METHOD

(71) Applicant: National Central University, Taoyuan (TW)

(72) Inventors: SHIH-CHING YEH, Taoyuan (TW); Hsiao-Kuang Wu, Taoyuan (TW)

(73) Assignee: National Central University, Taoyuan (TW)

(21) Appl. No.: 18/926,310

(22) Filed: Oct. 25, 2024

(30) Foreign Application Priority Data
Jul. 17, 2024 (TW) 113126693

(51) Int. Cl. G10L 25/63; G06V 40/16; G10L 15/26; G16H 10/20; G16H 20/70

(52) U.S. Cl. CPC G10L 25/63; G06V 40/174; G10L 15/26; G16H 10/20; G16H 20/70

(57) ABSTRACT

A detection system, comprising an interaction module, a receiving module, and an analysis module. The interaction module is configured to interact with a tested person, and the interaction module includes an audio acquisition unit to collect voice information emitted by the tested person. The receiving module is electrically connected to the interaction module to generate sound frequency data and speech text data based on the voice information obtained by the audio acquisition unit, and the analysis module is electrically connected to the receiving module. When the tested person responds to at least one question posed by the interaction module, causing the interaction module to generate voice information, the analysis module determines the emotional state of the tested person based on the sound frequency data, and assesses whether the tested person's response aligns with their emotional state based on the speech text data. If the tested person's response aligns with their emotional state, the response is judged as truthful; otherwise, it is judged as false.

### CROSS REFERENCE

The present invention claims priority to TW113126693, filed on Jul. 17, 2024.

### BACKGROUND OF THE INVENTION

### 1. Field of the Invention

This disclosure relates to an evaluation method, and in particular to an evaluation method for determining the authenticity of a tested person's answers to questions.

### 2. Description of the Related Art

Depression is a common mental health disorder, typically manifested by a persistent low mood, loss of interest or pleasure, along with a range of other physical and psychological symptoms. This condition can affect a person's emotions, behavior, and physical health. Depression not only causes distress for the individual but can also impact their work, studies, and interpersonal relationships.

However, conventional diagnostic questionnaires for depression have their limitations. The most common issue is that the results of questionnaire assessments are influenced by the patient's subjective reporting, which may be limited by the patient's memory, understanding, and evaluation abilities. Some individuals may not answer questions honestly, or they may overestimate or underestimate their symptoms. Therefore, determining the authenticity of a tested person responses to the questions becomes a critical issue.

### BRIEF SUMMARY OF THE INVENTION

The present disclosure provides A detection system, a host device, an evaluation method, and a computer-readable storage medium storing at least one program code, for identifying the authenticity of a tested person's answers to questions and further evaluating their emotional state.

The detection system provided in the present disclosure comprises an interaction module, a receiving module, and an analysis module. The interaction module is configured to interact with the tested person, and the interaction module includes an audio acquisition unit that can collect voice information emitted by the tested person. The receiving module is electrically connected to the interaction module to generate a sound frequency data and a speech text data based on the voice information obtained by the audio acquisition unit. The analysis module is electrically connected to the receiving module. When the tested person responds to at least one question posed by the interaction module, causing the interaction module to generate voice information, the analysis module can determine the tested person's emotional state based on the sound frequency data and assess whether the tested person's response is consistent with their emotional state based on the content of the speech text data. If the tested person's response aligns with his/her emotional state, the response is judged as truthful; otherwise, it is judged as false.

In some embodiments, the interaction module further includes an image capture module to collect image information of the tested person. When the receiving module obtains the image information from the interaction module, it generates facial expression data, eye movement data, and heart rate data based on the image information. If the analysis module is unable to determine the tested person's emotional state based on the sound data and speech text data, it will instead assess the tested person's emotional state using the facial expression data, eye movement data, and heart rate data.

The host device provided in the present disclosure comprises a connection module, a receiving module, and an analysis module. The connection module connects to a terminal device through wired or wireless means to obtain voice information generated by the terminal device from the speech of a tested person. The receiving module is electrically connected to the connection module and generates sound frequency data and speech text data based on the voice information. The analysis module is electrically connected to the receiving module and determines the tested person's emotional state based on the sound frequency data, while also assessing whether the content of the tested person's speech aligns with their emotional state based on the speech text data.

In some embodiments, the connection module further obtains image information generated by the terminal device from the captured images of the tested person. The receiving module then generates facial expression data, eye movement data, and heart rate data based on the image information. If the analysis module is unable to determine the tested person's emotional state based on the sound data and speech text data, it will assess the tested person's emotional state using the facial expression data, eye movement data, and heart rate data.

The evaluation method provided in the present disclosure includes capturing the speech of the tested person and generating sound frequency data and speech text data. Subsequently, the emotional state of the tested person is determined based on the sound frequency data, and it is assessed whether the tested person's speech aligns with their emotional state based on the speech text data. If so, the tested person's speech is judged as truthful; if not, it is judged as false.

In some embodiments, the evaluation method of the present disclosure further includes capturing the image of the tested person and generating facial expression data, eye movement data, and heart rate data. When the emotional state of the tested person cannot be determined based on the sound frequency data, the emotional state is assessed using the facial expression data, eye movement data, and heart rate data.

In some embodiments, the evaluation method of the present disclosure further includes assigning a plurality of weights respectively to the sound frequency data, facial expression data, eye movement data, and heart rate data. When the emotional state of the tested person cannot be determined based on the facial expression data, eye movement data, and heart rate data, a comprehensive analysis is performed using the sound frequency data, facial expression data, eye movement data, heart rate data, and their corresponding weights to determine the emotional state of the tested person.

The computer-readable storage medium provided in the present disclosure is applicable to a host device. When the program code stored in the medium is executed, the host device performs at least the following steps: connecting to a terminal device and enabling the terminal device to ask at least one question. When a tested person answers the at least one question, the voice information of the tested person is collected from the terminal device. Subsequently, sound frequency data and speech text data are generated based on the voice information. Furthermore, the present disclosure allows the emotional state of the tested person to be determined based on the sound frequency data and assesses whether the content of the tested person's answer aligns with their emotional state based on the speech text data. If the tested person's answer aligns with their emotional state, the content of their speech is judged as truthful; otherwise, it is judged as false.

In some embodiments, when the at least one program code is executed, it further controls the host device to perform at least the following steps: obtaining the image information generated by the terminal device from the captured image of the tested person, and generating facial expression data, eye movement data, and heart rate data based on the image information. When the emotional state of the tested person cannot be determined based on the sound data and speech text data, the emotional state is assessed using the facial expression data, eye movement data, and heart rate data.

In some embodiments, the system provided by the present disclosure further comprises a communication module, which is coupled to the controller, so as to transmit the plurality of physiological data on a network connection.

When determining the answers of the tested person's response, the host device is controlled to evaluate whether the emotional state of the tested person falls within a predefined range based on the at least one question, the content of the speech text, and the judgment result.

Since the present disclosure can determine whether the tested person's response aligns with their emotional state based on both voice information and image information, it allows for a more accurate evaluation of whether the tested person's emotional state falls within a predefined range.

Upon reviewing the following embodiments, those ordinarily skilled in the art will readily understand the underlying spirit of the present case, along with other inventive objectives, as well as the technical means and implementation methods employed in this case.

### BRIEF DESCRIPTION OF THE DRAWINGS

To make the aforementioned and other objectives, features, advantages, and embodiments of the present case more apparent and understandable, the attached drawings are described as follows:

FIG. 1 illustrates a block diagram of a detection system according to a first embodiment of the present disclosure.

FIG. 2 illustrates a functional block diagram of an interaction module according to one embodiment of the present disclosure.

FIG. 3 illustrates a schematic diagram of a virtual person according to one embodiment of the present disclosure.

FIG. 4 illustrates a block diagram of a detection system according to a second embodiment of the present disclosure.

FIG. 5 illustrates a block diagram of a host device according to one embodiment of the present disclosure.

FIG. 6A illustrates a flowchart of the steps of a depression evaluation method according to a first embodiment of the present disclosure.

FIG. 6B illustrates a flowchart of the steps of a depression evaluation method according to a second embodiment of the present disclosure.

FIG. 6C illustrates a flowchart of the steps of a depression evaluation method according to a third embodiment of the present disclosure.

### DETAILED DESCRIPTION OF THE INVENTION

To facilitate understanding of the object, characteristics and effects of this present disclosure, embodiments together with the attached drawings for the detailed description of the present disclosure are provided.

Unless otherwise defined in this specification, the meaning of scientific and technical terms used herein is consistent with the understanding and customary usage by those ordinarily skilled in the art to which this case pertains. Furthermore, unless context indicates otherwise, singular nouns used in this specification include their plural forms, and plural nouns include their singular forms.

Additionally, the terms “coupled” or “connected” as used herein may refer to two or more elements being in direct physical or electrical contact with each other, or in indirect physical or electrical contact with each other. These terms may also refer to two or more elements operating or functioning in cooperation with each other. Furthermore, they may refer to the interaction of two or more elements that are substantially connected or signal-connected.

In this document, the term “module” generally refers to an object comprising one or more transistors and/or one or more active or passive components connected in a specific manner to process signals.

Certain terms have been used in the specification and claims to refer to specific elements. However, those skilled in the art will understand that the same elements may be referred to by different names. The specification and claims do not distinguish elements based on name differences, but rather based on their functional differences. The term “comprising” as mentioned in the specification and claims is an open-ended term, and should be interpreted as “including but not limited to.”

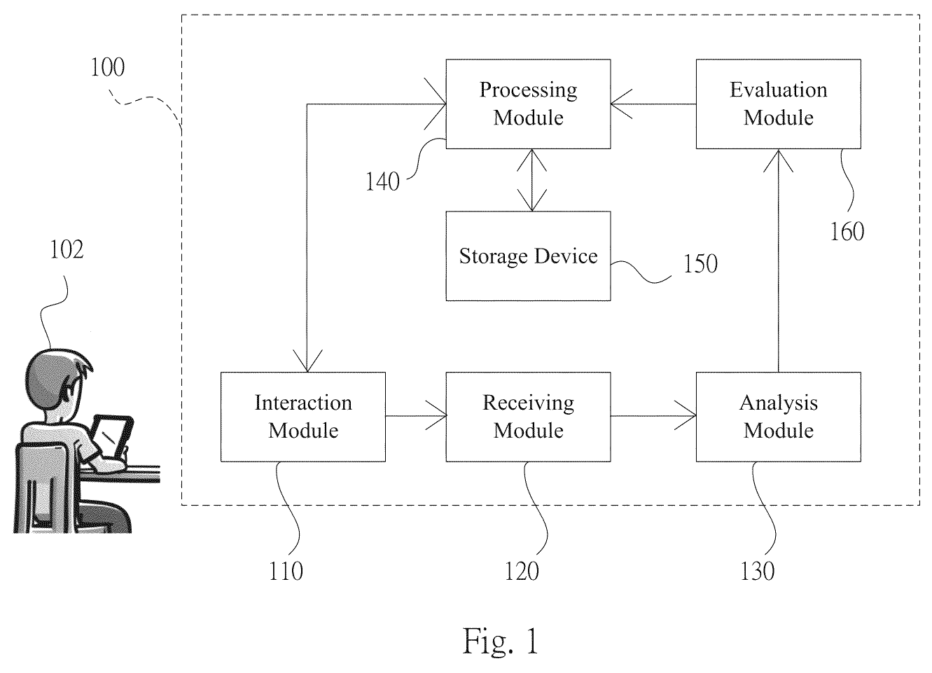

FIG. 1 illustrates a block diagram of a detection system according to a first embodiment of the present disclosure. Referring to FIG. 1, in this embodiment, the detection system 100 includes an interaction module 110, a receiving module 120, and an analysis module 130. The interaction module 110 is used to interact with a tested person 102, and the interaction module 110 can be coupled to the receiving module 120, while the receiving module 120 is coupled to the analysis module 130.

The detection system 100 may further include a processing module 140 and a storage device 150. The processing module 140 may be, for example, a central processing unit (CPU), graphics processing unit (GPU), embedded system, microcontroller, application-specific integrated circuit (ASIC), or the like, without limitation by the present disclosure. In addition, the storage device 150 may be a non-volatile memory such as a hard drive, flash memory, optical storage media, etc., capable of storing at least one program code. The processing module 140 is coupled to the storage device 150 to read the program code stored in the storage device 150.

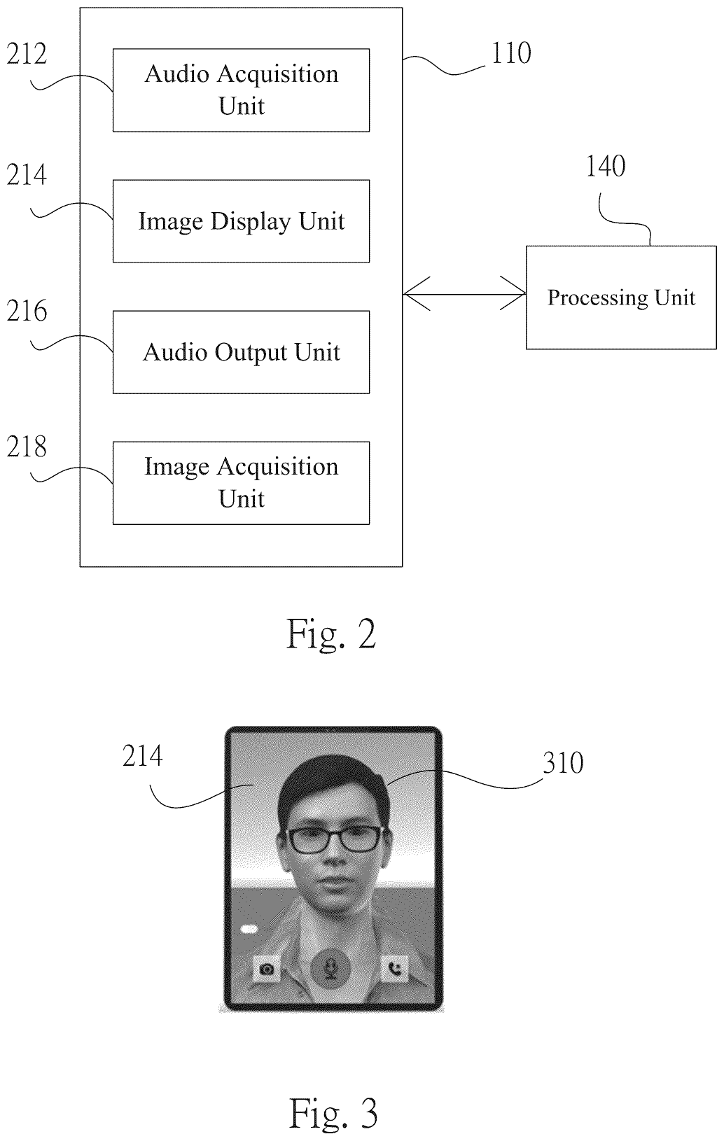

FIG. 2 illustrates a functional block diagram of an interaction module according to one embodiment of the present disclosure. Referring to FIG. 2, in this embodiment, the interaction module 110 includes at least an audio acquisition unit 212. In some embodiments, the audio acquisition unit 212 may be, for example, a microphone, but the present disclosure is not limited to this. In other embodiments, the interaction module 110 further includes an image display unit 214 and an audio output unit 216, such as a display panel and speakers; however, the present disclosure does not limit these examples. In these embodiments, the interaction module can be coupled to the processing unit 140.

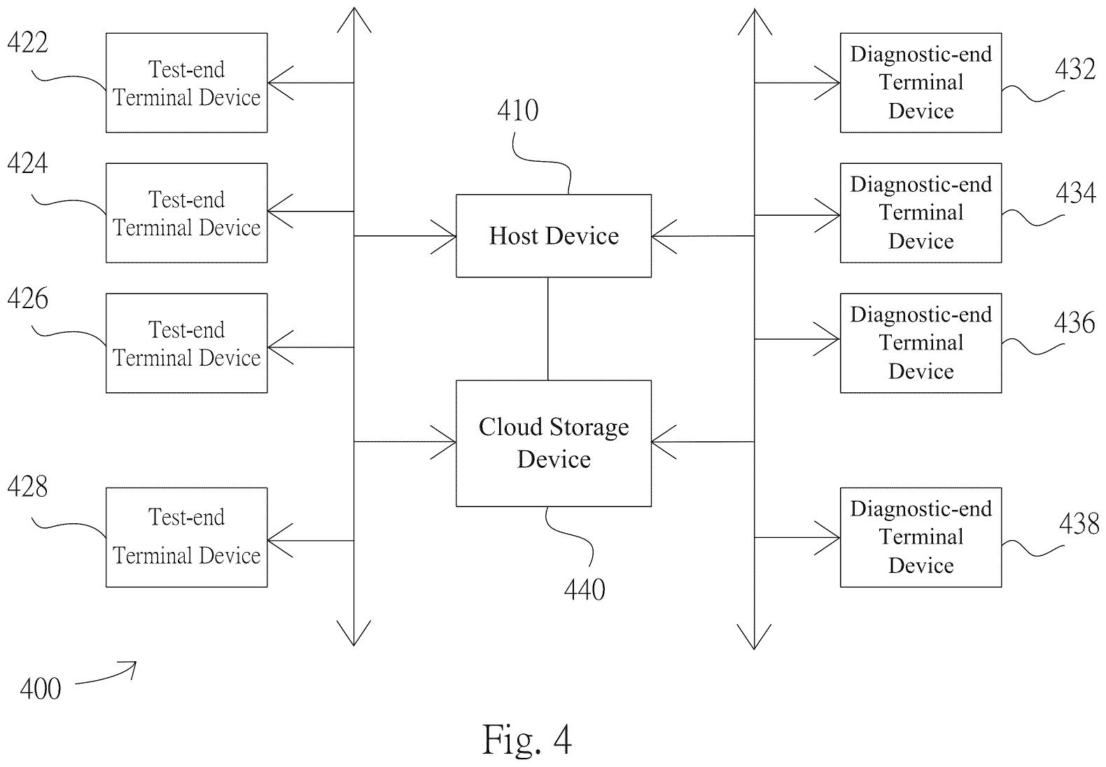

Referring to FIGS. 1 and 2, when the processing module 140 reads the program code from the storage device 150, it can cause the interaction module 110 to display a virtual character, such as the virtual counselor 310 shown in FIG. 3, via the image display unit 214. The interaction module 110 can also ask the tested person 102 at least one question through the audio output unit 216. For example, the virtual counselor 310 may ask the tested person 102, “Do you usually enjoy going out?” If the tested person 102 answers this question, the audio acquisition unit 212 can capture this voice information, and the processing module 140 will control the interaction module 110 to transmit the voice information to the receiving module 120.

When the receiving module 120 receives the voice information transmitted from the interaction module 110, it can separate the information into sound frequency data and speech text data. The sound frequency data can be an audio waveform signal, while the speech text data represents the content of the tested person's 102 response. For example, if the tested person 102 answers “Yes,” the receiving module 120 can recognize the words “Yes” as the speech text data. The receiving module 120 then transmits the sound frequency data and the speech text data to the analysis module 130 for further analysis.

In this embodiment, it is preferable for the receiving module 120 to process voice information using a Bidirectional Encoder Representations from Transformers (BERT) language model. The BERT model consists of a 12-layer transformer encoder with 12 bidirectional self-attention heads, containing a total of 110 million parameters. It provides word embeddings that may include contextual information. The analysis module 130 inputs the tokenized text into the pre-trained BERT model and selects the output from the last layer, which is a vector of length 768, as the textual feature representation. This vector is then treated as the speech text data and sent to the analysis module 130.

When the analysis module 130 receives the sound frequency data, it first extracts at least one acoustic feature. In this embodiment, the acoustic features extracted by the analysis module 130 include Mel Spectrogram, Mel-Frequency Cepstral Coefficients (MFCCs), Spectral Contrast, Chromagram, Tonal Centroid Features, and Tonnetz. The Mel Spectrogram converts the raw audio spectrogram (a heatmap describing the variation of frequency components over time) into the Mel scale, which is used to represent sound signal characteristics. MFCCs are widely used in speech recognition and voice identification because they reflect human auditory perception of different frequencies. Spectral Contrast features estimate the relative distribution of differences between spectral peaks and valleys in each sub-band, based on the representation method of musical octaves (also known as full octaves). The Chromagram is computed from the audio, projecting the full spectrum into 12 bins, each representing a different semitone in a musical octave. Tonnetz maps the 12-bin chroma vector into a 6-dimensional space capable of detecting harmonic changes. The analysis module 130 then concatenates several acoustic features into a 193-dimensional vector as audio features, which is treated as sound frequency data and sent to the analysis module 130.

Although the techniques for obtaining speech text data and sound frequency data have been disclosed above, those ordinarily skilled in the art will understand that the present disclosure is not limited to these methods.

When the analysis module 130 obtains the speech text data and sound frequency data, it can determine whether the content of the speech text data aligns with the emotional state of the tested person 102. If they are consistent, the tested person's response is judged as truthful; otherwise, the response is judged as false.

Referring again to FIG. 1 and FIG. 2, in some other embodiments, the interaction module 110 further includes an image acquisition unit 218, such as a camera. However, the present disclosure is not limited to this. When the analysis module 130 is unable to determine the emotional state of the tested person 102 based on the sound frequency data, the processing module 140 can cause the image acquisition unit 218 to capture image information of the tested person 102 and transmit it to the receiving module 120.

When the receiving module 120 receives the image information of the tested person, it can extract facial expression data, eye movement data, and heart rate data from the information, and transmit these data to the analysis module 130 for further analysis.

Eye movement reflects the cognitive processing demands in our brain. Therefore, the present disclosure utilizes eye movement data as a feature model for psychological assessment. First, the receiving module 120 can use technologies such as Unity ARKit to obtain the gaze point from the image information, which helps track the point of focus on the screen. Second, the receiving module 120 can calculate gaze duration, saccades, and event statistics. It calculates the mean, standard deviation, and maximum value for gaze duration, the mean and standard deviation for saccades, and the fixation rate and saccade rate from event statistics. In total, seven eye movement features are calculated and treated as eye movement data, which are then sent to the analysis module 120.

Additionally, in this embodiment, the term “heart rate data” may refer to heart rate variability (HRV); however, the present disclosure is not limited to this. Heart rate variability (HRV) refers to the variation in time intervals between consecutive heartbeats. Changes in the sympathetic and parasympathetic nervous systems affect heart rate. The function of the sympathetic nervous system can be summarized by the 3Fs: Fight, Flight, Fright, or Sex. The sympathetic nervous system is stimulated in emergency states (the 4Es: Emergency, Embarrassment, Excitement, and Exercise). Therefore, HRV varies depending on the physical and psychological state of the body, and it serves as a clinical indicator of psychological well-being.

The technique for obtaining heart rate data from image information has been documented in various sources. For example, China Patent Application CN201510741006.9A discloses a non-contact heart rate detection method. However, those skilled in the art will understand that different techniques using cameras to detect heart rate do not affect the fundamental spirit of the present invention. Specifically, in this embodiment, the receiving module 120 obtains heart rate data from the image information using remote photoplethysmography (rPPG).

Remote photoplethysmography (rPPG) is a non-contact, video-based method that monitors changes in blood volume by capturing variations in pixel intensity from the skin to measure pulse rate. Heartbeats influence blood flow, which in turn causes subtle changes in skin brightness, allowing us to estimate heart rate using this clue. In this embodiment, when the image information is sent to the analysis module 120, the analysis module 120 uses an HRV analysis toolkit to measure 23 HRV indicators in both the time domain and frequency domain, which are then treated as heart rate data and sent to the analysis module 130.

When the analysis module 130 receives the facial data, eye movement data, and heart rate data, it can determine the emotional state of the tested person 102 based on these data. Conversely, if the analysis module 130 is still unable to analyze the emotional state of the tested person 102, it can perform a comprehensive analysis of the received speech text data, sound frequency data, facial data, eye movement data, and heart rate data. For example, by assigning different weight values to each type of data, a comprehensive analysis is conducted based on the speech text data, sound frequency data, facial data, eye movement data, and heart rate data along with their respective weight values to determine the emotional state of the tested person 102.

In this embodiment, the analysis module 130 employs various machine learning algorithms, including K-Nearest Neighbors (KNN), Support Vector Machines (SVM), Decision Trees (DT), Random Forests (RF), Multilayer Perceptrons (MLP), Adaptive Boosting (AdaBoost), and Gradient Boosting (GB) to build multiple unimodal classifiers for analyzing the speech text data, sound frequency data, heart rate data, and eye movement data. Additionally, since the facial data obtained from the image information represent time-sequential facial features of the tested person, this embodiment uses Long Short-Term Memory (LSTM) with a Deep Neural Network (DNN) for facial data analysis.

In some embodiments, the analysis module 130 uses the Facial Attribute Network (FAb-Net) to analyze facial data. The Facial Attribute Network is a self-supervised framework designed to learn facial attribute embeddings that encode details about head pose, facial landmarks, and facial expressions. FAb-Net has been trained on a dataset containing a vast number of conversation segments, even exceeding millions of dialogue clips, involving thousands of speakers. The network encodes facial expressions, head pose, and other facial attributes from conversations with users. After cropping each frame to fit the face, the frames are input into the model to obtain a 256-length facial embedding. Since the duration of each video clip varies, an LSTM layer is added to unify all facial embeddings into a fixed length. This process corresponds to the aforementioned analysis of facial data using LSTM with DNN.

In some embodiments, the analysis module 130 employs a deep neural network (DNN) to integrate the data transmitted by the receiving module 120. For example, with facial data, since the time required for the tested person 102 to answer each question varies, the length of the facial feature embeddings also differs. An LSTM layer is added to extract key information from the facial features and standardize the dimension of the facial embeddings. After the facial embeddings pass through the LSTM layer, all feature vectors are concatenated together as input and fed into a deep neural network with two hidden layers. Following the first hidden layer, there is a dropout layer with a dropout rate of 0.2, containing 512 neurons, with L2 regularization applied. The second hidden layer contains 256 neurons, followed by another dropout layer with a dropout rate of 0.2. The deep neural network with feature-level fusion is trained for 100 epochs, using the Adam optimizer with a learning rate of 0.001. This approach enables the accurate analysis of the truthfulness of the tested person's 102 responses to the questions.

Referring again to FIG. 1, in some other embodiments, the detection system 100 further includes an evaluation module 160, which is coupled to the analysis module 130. This allows the evaluation module 160 to assess whether the emotional state of the tested person 102, based on the analysis results from the analysis module 130, falls within a predefined range. More specifically, the evaluation module 160 can assess the probability of the tested person's 102 level of depression, classifying it into categories such as healthy, mild, moderate, severe depression, or bipolar disorder. In other embodiments, the analysis module 130 may be coupled to the processing module 140, which can transmit the evaluation results to the processing module 140. The processing module 140 can then send the results to the interaction module 110 to report them to the tested person 102. For example, the virtual counselor 310 can inform the tested person 102 of the evaluation results.

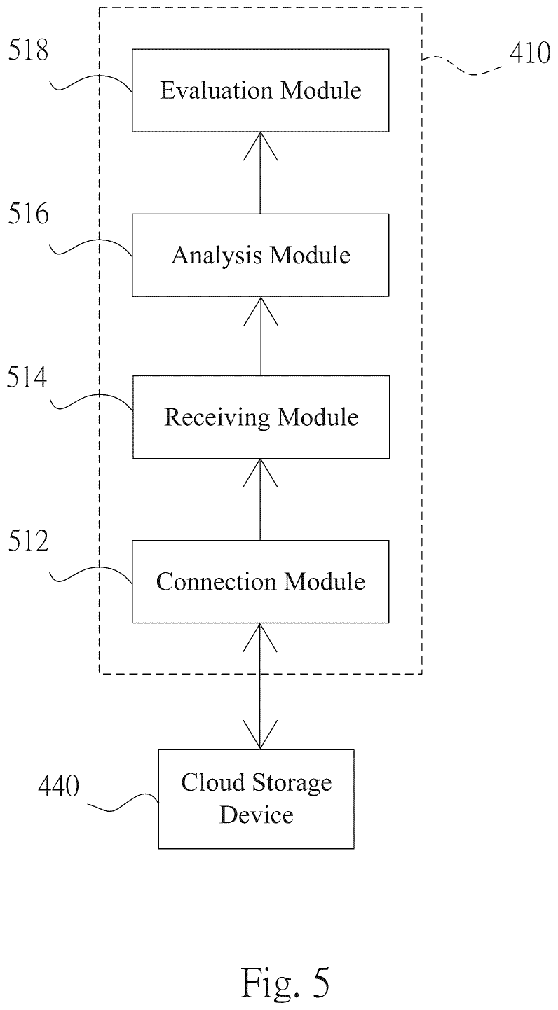

FIG. 4 illustrates a block diagram of a detection system according to a second embodiment of the present disclosure. Referring to FIG. 4, the detection system 400 provided in this embodiment includes a host device 410, which is coupled to at least one test-end terminal device 422 via wired means, such as Ethernet cables, coaxial cables, fiber optics, USB cables, or via wireless means, such as wireless networks, mobile communication networks, Bluetooth, and other wireless protocols. In this embodiment, the test-end terminal device 422 may be, for example, a smartphone, tablet, industrial computer, desktop computer, or laptop, without limitation to the present disclosure. In some embodiments, the host device 410 can be coupled to multiple test-end terminal devices, such as 424, 426, and 428.

FIG. 4 illustrates a block diagram of a host device according to one embodiment of the present disclosure. Referring to FIG. 4, the host device 410 includes a connection module 512, a receiving module 514, and an analysis module 516. The connection module 512 can be coupled to the receiving module 514, and the receiving module 514 can be coupled to the analysis module 516. In this embodiment, the connection module 512 may vary depending on the interface used to connect with the test-end terminal device. It could be different types of connection ports, such as an Ethernet port or a USB port, or it could be an interface card, such as a wireless network card or other network cards, without limitation to the present disclosure.

Referring to both FIGS. 3 and 4, the test-end terminal devices 422, 424, 426, and 428 each have a screen, an audio output unit, an audio acquisition unit, and an image acquisition unit (not shown), similar to the interaction module 110 in FIG. 1. For the sake of explanation, the following description will use the test-end terminal device 422 as an example, and those skilled in the art can apply the same principles to the other test-end terminal devices.

In this embodiment, the test-end terminal device 422 can replace the interaction module 110 from FIG. 1 by displaying the virtual counselor 310 on its screen to interact with a tested person and ask pre-set questions. When the tested person responds, the test-end terminal device 422 can collect the tested person's voice information and capture their image information. The test-end terminal device 422 can then transmit the collected voice and image information to the connection module 512.

When the connection module 512 obtains the voice and image information from the test-end terminal device 422, it can transmit this information to the receiving module 514. Similar to the receiving module 120 in FIG. 1, the receiving module 514 can extract speech text data and sound frequency data from the voice information. Additionally, the receiving module 514 can extract eye movement data, facial data, and heart rate data from the image information. The receiving module 514 then sends the obtained data to the analysis module 516. The analysis module 516, like the analysis module 130 in FIG. 1, analyzes the data sent by the receiving module 514 to determine whether the tested person's response aligns with their emotional state. If the response is judged to align with the emotional state, it is considered truthful; otherwise, it is considered false. The detailed process of this judgment has been thoroughly described in the discussion of FIG. 1 and will not be repeated here.

Additionally, in some embodiments, the host device 410 may also be configured with an evaluation module 518, which is coupled to the analysis module 516. The analysis module 516 can send its judgment results to the evaluation module 518, which can provide an assessment similar to the evaluation module 160 in FIG. 1. In other embodiments, the evaluation module 518 may be coupled to the connection module 512.

In other embodiments, the connection module 512 may also connect to at least one diagnostic-end terminal device 432 via the aforementioned wired or wireless methods. The diagnostic-end terminal device 432 may be the same or similar to the test-end terminal device 422, and further details will not be repeated here. Additionally, in some embodiments, the connection module 512 can connect to multiple diagnostic-end terminal devices, such as 432, 434, 436, and 438, without limitation in the present disclosure. For simplicity, the following description will use diagnostic-end terminal device 432 as an example, and those skilled in the art can apply the same principles to other diagnostic-end terminal devices.

When the evaluation module 518 generates an evaluation report, it can be transmitted to the test-end terminal device 422 through the connection module 512 to provide feedback to the tested person. In other embodiments, the evaluation module 518 can send its evaluation report to the diagnostic-end terminal device 432 via the connection module 512. This allows a diagnostician, such as a psychologist, to use the evaluation report to diagnose and treat the tested person.

In this embodiment, the host device 410 is connected in parallel to a plurality of test-end terminal devices 422, 424, 426, and 428, allowing for the evaluation of different tested persons and the generation of corresponding evaluation reports. Additionally, the host device 410 can also be connected in parallel to multiple diagnostic-end terminal devices 432, 434, 436, and 438. This allows the evaluation reports generated by the evaluation module 518 to be sent to one or more of the diagnostic-end terminal devices 432, 434, 436, or 438. In some embodiments, each test-end terminal device 422, 424, 426, and 428 is corresponding to one of the diagnostic-end terminal devices 432, 434, 436, or 438 respectively, ensuring that the evaluation report of each tested person is sent to the corresponding diagnostic-end terminal device.

In other embodiments, the connection module 512 can also be coupled to a cloud storage device 440. In these embodiments, the evaluation module 518 can store the generated evaluation reports in the cloud storage device 440. This allows diagnosticians to access the corresponding evaluation reports of tested persons by connecting to the cloud storage device 440 through any of the test-end terminal devices 422, 424, 426, or 428. Of course, those skilled in the art will understand that the cloud storage device 440 can be replaced by local storage of the host device 410 without affecting the core spirit of the present invention.

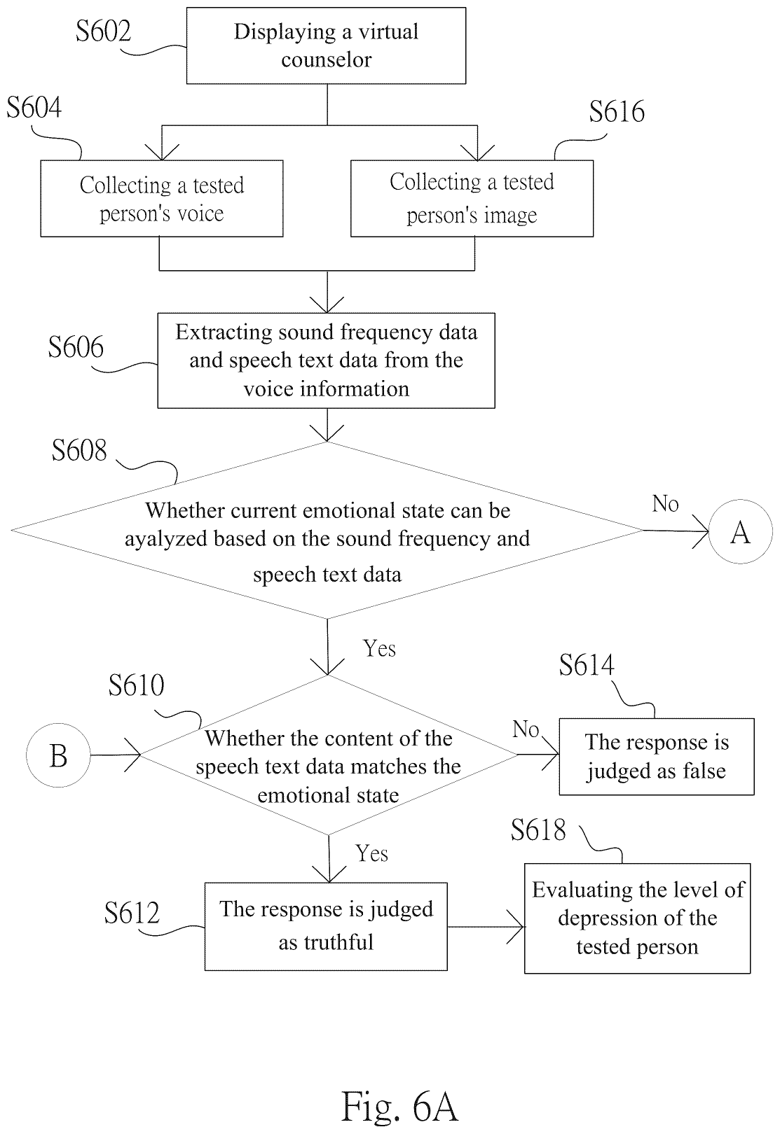

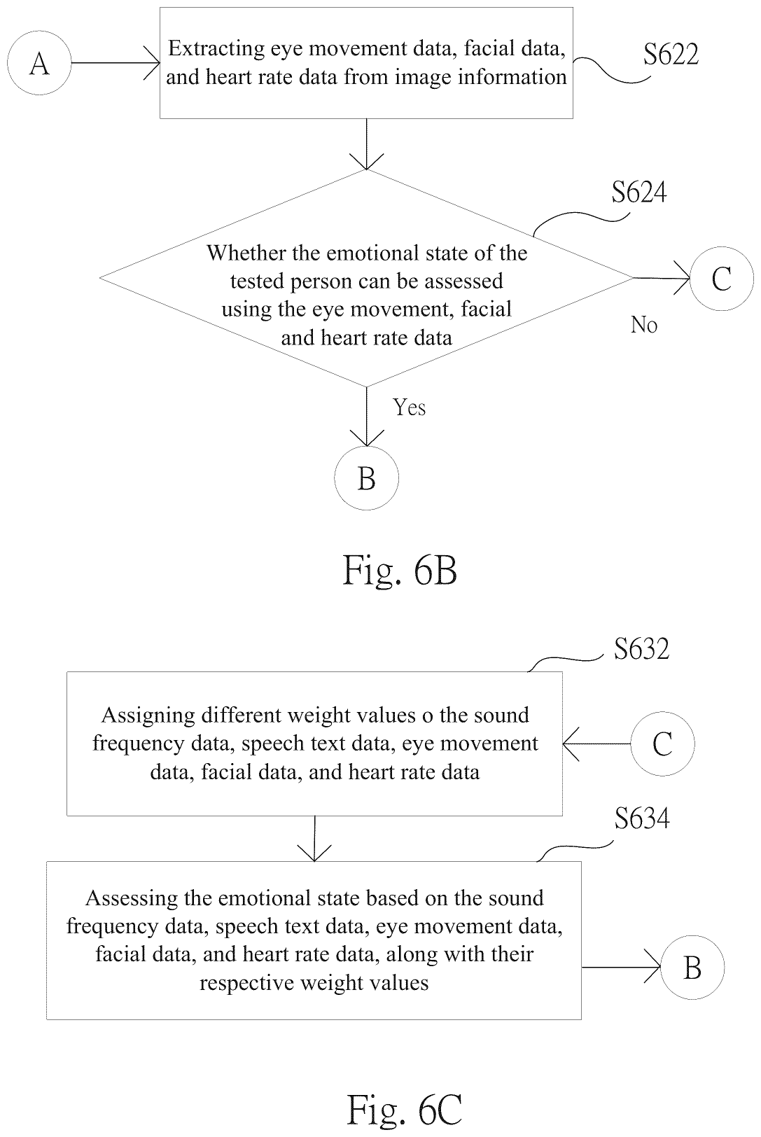

FIG. 5A illustrates a flowchart of the steps of a depression evaluation method according to a first embodiment of the present disclosure. Referring to FIG. 5A, the evaluation method provided in this embodiment includes step S602, where a character is displayed as a virtual counselor, and questions from a question bank are asked to a tested person. When the tested person responds, as described in step S604, the tested person's voice is collected, and voice information is generated. Then, step S606 is performed, where sound frequency data and speech text data are extracted from the voice information. This allows for step S608, where the current emotional state of the tested person is analyzed based on the sound frequency data and speech text data.

If the emotional state of the tested person is analyzed in step S608, proceed to step S610, where it is determined whether the content of the speech text data matches the emotional state. If they match, the tested person's response is judged as truthful in step S612. If they do not match, the response is judged as false in step S614.

Referring further to FIG. 5A, when the tested person responds to the virtual counselor's questions, this embodiment can also perform step S616, where the image information of the tested person is captured. Thus, as described in step S618, this embodiment can evaluate the level of depression of the tested person based on both the voice information and the image information.

FIG. 5B illustrates a flowchart of the steps of a depression evaluation method according to a second embodiment of the present disclosure. Referring to FIG. 5B, if in step S608 of FIG. 5A, the emotional state of the tested person cannot be determined using the sound frequency data and speech text data, step S622 is executed, where eye movement data, facial data, and heart rate data are extracted from the image information generated in step S616 of FIG. 5A. Next, as described in step S624, it is determined whether the emotional state of the tested person can be assessed using the eye movement data, facial data, and heart rate data. If the emotional state is determined in step S624, the process can return to step S610 of FIG. 5A.

FIG. 5C illustrates a flowchart of the steps of a depression evaluation method according to a third embodiment of the present disclosure. Referring to FIG. 5C, if in step S624 of FIG. 5B, the emotional state of the tested person cannot be determined using the eye movement data, facial data, and heart rate data, then step S632 is executed, where different weight values are assigned to the sound frequency data, speech text data, eye movement data, facial data, and heart rate data. Next, as described in step S634, the emotional state of the tested person is comprehensively assessed based on the sound frequency data, speech text data, eye movement data, facial data, and heart rate data, along with their respective weight values, and the process returns to step S610 of FIG. 5A.

As seen from FIG. 5A to FIG. 5B, the present disclosure employs a three-stage method to analyze the emotional state of the tested person. Compared to conventional methods that rely on a single approach, such as using only eye movement or heart rate to assess the tested person's emotions, the present disclosure offers higher accuracy and a more reliable analysis of the tested person's emotional state.

Additionally, the present disclosure uses algorithms to determine whether the speech text data of the tested person aligns with their emotional state, thereby verifying the truthfulness of the tested person's responses. This enables the present disclosure to more accurately assess the depression status of the tested person.

While the present disclosure has been described by means of specific embodiments, numerous modifications and variations could be made thereto by those skilled in the art without departing from the scope and spirit of the present disclosure set forth in the claims.

What is claimed is:

1. A detection system, comprising:
an interaction module, configured to interact with a tested person, the interaction module having an audio acquisition unit to collect voice information emitted by the tested person;
a receiving module, electrically connected to the interaction module, to generate sound frequency data and speech text data based on the voice information obtained by the audio acquisition unit; and
an analysis module, electrically connected to the receiving module,
wherein when the tested person responds to at least one question posed by the interaction module, causing the interaction module to generate the voice information, the analysis module determines the emotional state of the tested person based on the sound frequency data, and assesses whether the content of the tested person's response aligns with their emotional state based on the speech text data. If the tested person's response aligns with their emotional state, the response is judged as truthful; otherwise, it is judged as false.

2. The detection system according to claim 1, wherein the interaction module further comprises an image acquisition module to collect image information of the tested person,
wherein when the receiving module obtains the image information from the interaction module, it generates facial expression data, eye movement data, and heart rate data based on the image information, and
wherein when the analysis module is unable to determine the emotional state of the tested person based on the sound data and speech text data, it determines the emotional state of the tested person based on the facial expression data, eye movement data, and heart rate data.

3. The detection system according to claim 2, wherein the analysis module further assigns multiple weight values respectively to the sound frequency data, the speech text data, the facial expression data, the eye movement data, and the heart rate data,
wherein when the analysis module is unable to determine the emotional state of the tested person based on the facial expression data, the eye movement data, and the heart rate data, a comprehensive analysis is performed based on the sound frequency data, the speech text data, the facial expression data, the eye movement data, and the heart rate data along with their corresponding weight values to determine the emotional state of the tested person.

4. The detection system according to claim 2, wherein the interaction module further comprises a display unit and an audio output unit, and the detection system further comprises:
a storage device, storing at least one program code; and
a processing module, coupled to the interaction module and the storage device, wherein when the processing module reads the at least one program code from the storage device, it executes the following steps:
displaying a virtual character on the display unit and enabling the virtual character to ask the at least one question through the audio output unit; and
when the tested person answers the at least one question, the processing module transmits the voice information collected by the audio acquisition unit and the image information collected by the image acquisition unit to the receiving module.

5. The detection system according to claim 2, further comprising:
an evaluation module, electrically connected to the analysis module,
wherein when the analysis module determines the truthfulness of the tested person's response, the determination result is transmitted to the evaluation module, enabling the evaluation module to assess whether the emotional state of the tested person falls within a predefined range based on the at least one question, the speech text content, and the determination result.

6. A host device, comprising:
a connection module, configured to connect to a terminal device through wired or wireless means, to obtain voice information generated by the terminal device from the speech of a tested person;
a receiving module, electrically connected to the connection module, to generate sound frequency data and speech text data based on the voice information; and
an analysis module, electrically connected to the receiving module, to determine the emotional state of the tested person based on the sound frequency data, and to assess whether the content of the tested person's speech aligns with their emotional state based on the speech text data,
wherein if the content of the tested person's speech aligns with their emotional state, the speech content is judged as truthful; otherwise, it is judged as false.

7. The host device according to claim 6, wherein the connection module further obtains image information generated by the terminal device from the captured image of the tested person,
wherein the receiving module generates facial expression data, eye movement data, and heart rate data based on the image information, and
wherein when the analysis module is unable to determine the emotional state of the tested person based on the sound data and speech text data, the emotional state is determined based on the facial expression data, eye movement data, and heart rate data.
an analysis module, electrically connected to the receiving module, to determine the emotional state of the tested person based on the sound frequency data, and to assess whether the content of the tested person's speech aligns with their emotional state based on the speech text data,
wherein if the content of the tested person's speech aligns with their emotional state, the speech content is judged as truthful; otherwise, it is judged as false.

8. The host device according to claim 7, wherein the analysis module further assigns multiple weight values respectively to the sound frequency data, speech text data, facial expression data, eye movement data, and heart rate data,
wherein when the analysis module is unable to determine the emotional state of the tested person based on the facial expression data, eye movement data, and heart rate data, a comprehensive analysis is performed based on the sound frequency data, speech text data, facial expression data, eye movement data, and heart rate data along with their corresponding weight values to determine the emotional state of the tested person.

9. The host device according to claim 6, further comprising:
an evaluation module, electrically connected to the analysis module,
when the analysis module verifies the truthfulness of the tested person's speech content, the determination result is transmitted to the evaluation module, allowing the evaluation module to assess whether the emotional state of the tested person falls within a predefined range based on the at least one question, the speech text content, and the determination result.

10. An evaluation method for determining whether the speech content of a tested person is truthful, comprising the following steps:
collecting the speech of the tested person and generating sound frequency data and speech text data; and
determining the emotional state of the tested person based on the sound frequency data, and evaluating whether the speech content of the tested person aligns with their emotional state based on the speech text data, wherein if the speech aligns with the emotional state, the speech is judged as truthful, and if not, it is judged as false.

11. The evaluation method according to claim 10, further comprising the following steps:
collecting the image of the tested person and generating facial expression data, eye movement data, and heart rate data; and
when the emotional state of the tested person cannot be determined based on the sound frequency data, determining the emotional state of the tested person based on the facial expression data, eye movement data, and heart rate data.

12. The evaluation method according to claim 11, further comprising the following steps:
assigning multiple weight values respectively to the sound frequency data, speech text data, facial expression data, eye movement data, and heart rate data; and
when the emotional state of the tested person cannot be determined based on the facial expression data, eye movement data, and heart rate data, performing a comprehensive analysis based on the sound frequency data, speech text data, facial expression data, eye movement data, and heart rate data along with their corresponding weight values to determine the emotional state of the tested person.

13. The evaluation method according to claim 11, further comprising the following steps:
displaying a virtual character on a terminal device and enabling the virtual character to ask the at least one question; and
when the tested person responds to the at least one question, controlling the terminal device to collect the tested person's speech to generate the sound frequency data and speech text data, and capturing the tested person's image to generate the facial expression data, eye movement data, and heart rate data.

14. A computer-readable storage medium, applicable to a host device, storing at least one program code, wherein when the program code is read, it controls the host device to perform at least the following steps:
linking to a terminal device and enabling the terminal device to ask at least one question;
when a tested person responds to the at least one question, collecting the tested person's voice information from the terminal device;
generating sound frequency data and speech text data based on the voice information;
determining the emotional state of the tested person based on the sound frequency data, and assessing whether the tested person's response aligns with their emotional state based on the speech text data and its content; and
if the tested person's response aligns with their emotional state, determining the response as truthful, otherwise, determining it as false.

15. The computer-readable storage medium according to claim 14, wherein when the program code is read, it further controls the host device to perform at least the following steps:
obtaining the image information generated by the terminal device from the captured image of the tested person;
generating facial expression data, eye movement data, and heart rate data based on the image information; and
when the emotional state of the tested person cannot be determined based on the sound data and speech text data, determining the emotional state of the tested person based on the facial expression data, eye movement data, and heart rate data.

16. The computer-readable storage medium according to claim 15, wherein when the program code is read, it further controls the host device to perform at least the following steps:
assigning multiple weight values respectively to the sound frequency data, speech text data, facial expression data, eye movement data, and heart rate data; and
when the emotional state of the tested person cannot be determined based on the facial expression data, speech text data, eye movement data, and heart rate data, performing a comprehensive analysis based on the sound frequency data, facial expression data, eye movement data, heart rate data, and their corresponding weight values to determine the emotional state of the tested person.

17. The computer-readable storage medium according to claim 14, wherein when the program code is read, it further controls the host device to perform at least the following steps:
when determining the truthfulness of the tested person's response, evaluating whether the emotional state of the tested person falls within a predefined range based on the at least one question, the speech text content, and the determination result.
